(19) United States
(12) Patent Application Publication
(10) Pub. No.: US 2011/0112428 A1
(43) Pub. Date: May 12, 2011

(54) Body composition measuring apparatus using a bioelectric impedance analysis associated with a neural network algorithm

(75) Inventors: Kuen-Chang Hsieh, Taichung County (TW); Liao-Chuan Chang, Taichung County (TW); Chih-Cheng Chao, Taichung County (TW)

(73) Assignee: CHARDER ELECTRONIC CO., LTD., TAICHUNG COUNTY (TW)

(21) Appl. No.: 12/656,217

(22) Filed: Jan. 21, 2010

(30) Foreign Application Priority Data
Nov. 9, 2009 (TW) 98137979

(51) Int. Cl. A61B 5/053

(52) U.S. Cl. 600/547

(57) ABSTRACT

A body composition measuring apparatus using a bioelectric impedance analysis and a neural network algorithm for obtaining two or more anthropometry variables from testees and then inputting the anthropometry variables into the internal processing unit that has a built-in back propagation-artificial neural network that has one input layer, 1-10 hidden layers each having 1-15 hidden neurons and one output layer having one output neuron. By means of the aforesaid artificial neural network, the invention accurately predict the fat free mass of the testee so as to further obtain the amount of body fat, showing higher accuracy than conventional linear regression equation (LRE).

### BACKGROUND OF THE INVENTION

1. Field of the Invention

The present invention relates to apparatus for measuring the body composition of a person and more particularly, to a body composition measuring apparatus using a bioelectric impedance analysis associated with a neural network algorithm.

2. Description of the Related Art

Conventional human body composition measuring apparatus using a bioelectric impedance analysis (BIA) commonly achieve prediction by means of a processor having a built-in linear regression equation (LRE). The linear regression equation is obtained by means of employing a linear regression analysis after collection of a predetermined number of human body information and anthropometry variables. In actual use of the apparatus, it is necessary to input or measure the anthropometry variables (such as, height, weight, bioelectrical impedance values, etc.) of the testee, so that the body composition (for example, body fat) of the testee can be predicted.

Although there is a direct correlation between the body composition and the anthropometry variables of body height, body weight, bioelectrical impedance values and etc., the correlation is non-linear. Therefore, using a linear regression analysis to predict the body composition of a person cannot accurately describe the body composition of the person, limiting the accuracy of the prediction.

Taiwan Patent 1291867, issued to the present inventor in 2003, discloses a human body composition neural network model. This patent discloses the concept of the use of an artificial neural network to predict the body composition of a human being based on the understanding that an artificial neural network was a non-linear dynamic system having adaptive resonance learning and fault-tolerance characteristics, the inventor thought the use of a neural network to predict the body composition of a human being were workable theoretically and proposed the aforesaid invention, and therefore inventor didn't disclose any example in the said patent but list roughly some neural network models with the objective of protecting the concept of using a neural network to predict human body composition. Right up till now, after several years of continuous study and verification, a high-precision neural network model for use in a body composition measuring apparatus is finally created, showing high precision.

### SUMMARY OF THE INVENTION

The present invention has been accomplished under the circumstances in view. It is the main object of the present invention to provide a body composition measuring apparatus using a bioelectric impedance analysis associated with a neural network algorithm, which shows a measuring precision higher than the bioelectric impedance analysis associated with the conventional linear regression equation.

To achieve this and other objects of the present invention, a body composition measuring apparatus comprises an apparatus body and a processing unit. The apparatus body comprises at least one anthropometry variables acquiring means for obtaining anthropometry variables of a testee by means of an inputting or measuring technique, including at least two of the age, body height, body weight and bioelectrical impedance values of the testee. The processing unit is mounted inside the apparatus body and connected to the at least one anthropometry variables acquiring means. The processing unit has built therein at least one back propagation-artificial neural network (BP-ANN). Each back propagation-artificial neural network comprises an input layer, 1-10 hidden layers, and an output layer. The input layer comprises a plurality of input neurons adapted for receiving the anthropometry variables from the at least one anthropometry variables acquiring means. Further, each hidden layer comprises 1-15 hidden neurons and a transfer function corresponding to each hidden neuron. Each transfer function can be a Log-Sigmoid or Hyperbolic Tangent Sigmoid. The output layer comprises an output neuron and a linear transfer function for outputting fat free mass (FFM) that can be processed by the processing unit to obtain the amount and ratio of the body fat the testee carries.

### BRIEF DESCRIPTION OF THE DRAWINGS

FIG. 1 is a system block diagram of a body composition measuring apparatus in accordance with the present invention.

FIG. 2 is a male fat free mass standard deviation (in comparison with the FFM measured by DEXA) interrelationship diagram obtained from the prediction of the preferred embodiment of the invention and the prediction of the conventional linear regression equation.

FIG. 3 is a female fat free mass standard deviation (in comparison with the FFM measured by DEXA) interrelationship diagram obtained from the prediction of the preferred embodiment of the invention and the prediction of the conventional linear regression equation.

FIG. 4 is a male fat free mass standard deviation (in comparison with the FFM measured by DEXA) distribution diagram obtained from the prediction of the preferred embodiment of the invention and the prediction of the conventional linear regression equation.

FIG. 5 is a female fat free mass standard deviation (in comparison with the FFM measured by DEXA) distribution diagram obtained from the prediction of the preferred embodiment of the invention and the prediction of the conventional linear regression equation.

FIG. 6 shows each measured standard deviation (in comparison with the FFM measured by DEXA) of the preferred embodiment of the present invention having 1-5 hidden layers and 1-10 hidden neurons.

FIGS. 7(A)-7(J) show the bioelectrical impedance values measuring methods and locations of the preferred embodiment of the present invention.

FIG. 8 shows every standard deviation (in comparison with the FFM measured by DEXA) of the whole body and each limb measured by the preferred embodiment of the present invention.

### DETAILED DESCRIPTION OF THE INVENTION

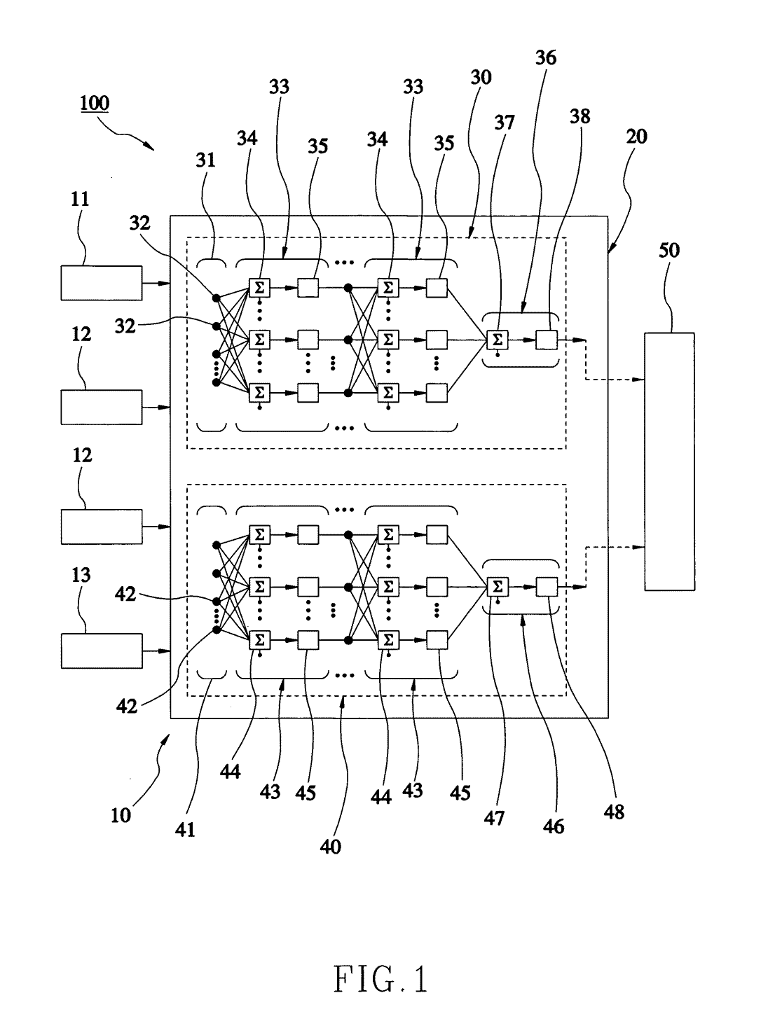

FIG. 1 is a system block diagram of a body composition measuring apparatus using a bioelectric impedance analysis processing with a neural network algorithm in accordance with the present invention. As illustrated, the body composition measuring apparatus 100 comprises an apparatus body 10, a processing unit 20 and a display unit 50.

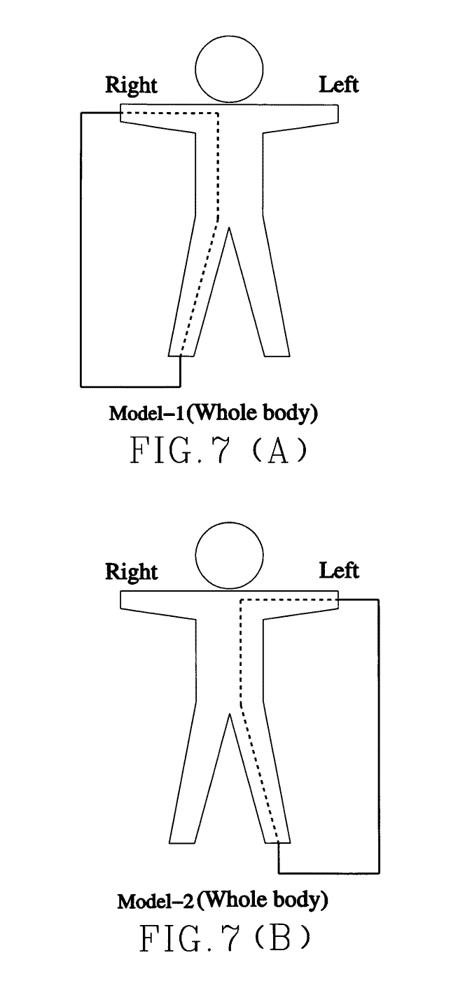

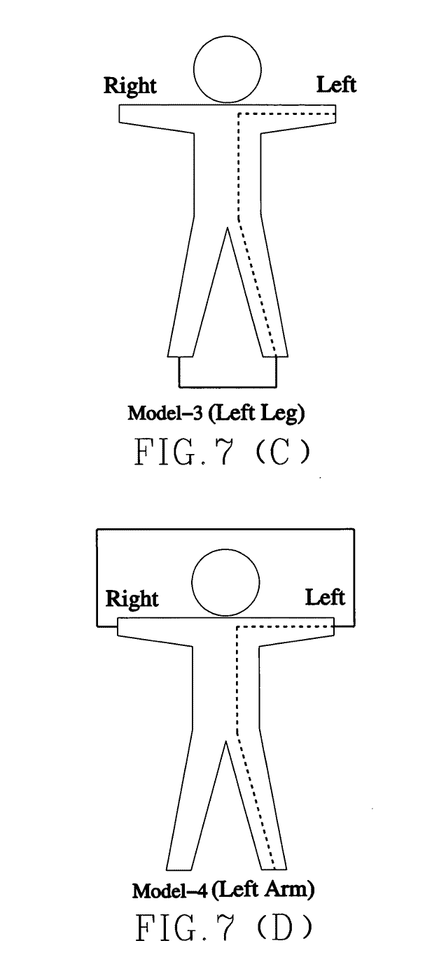

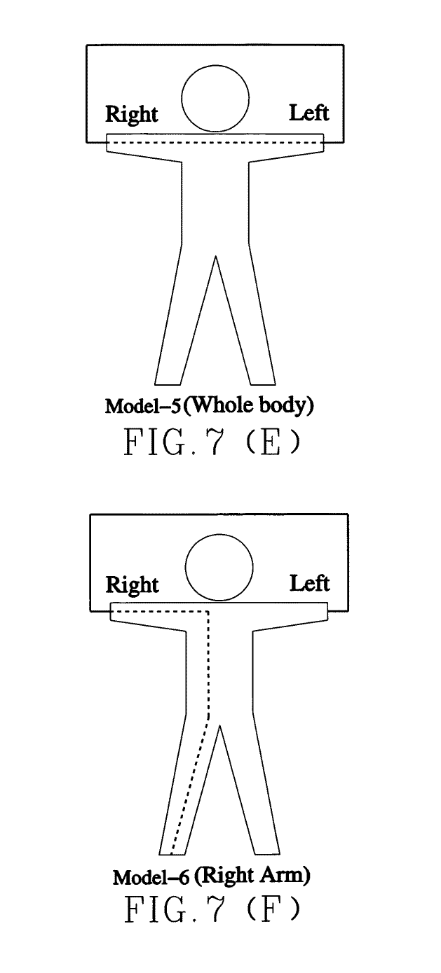

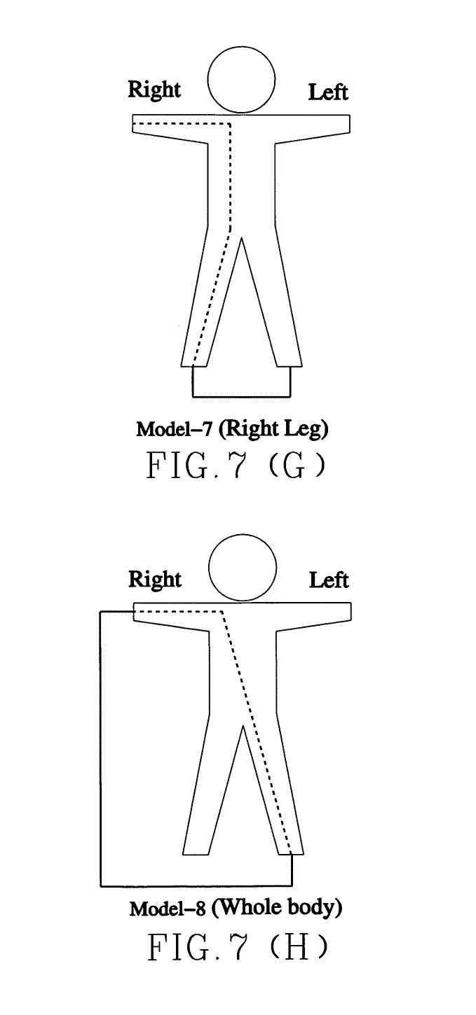

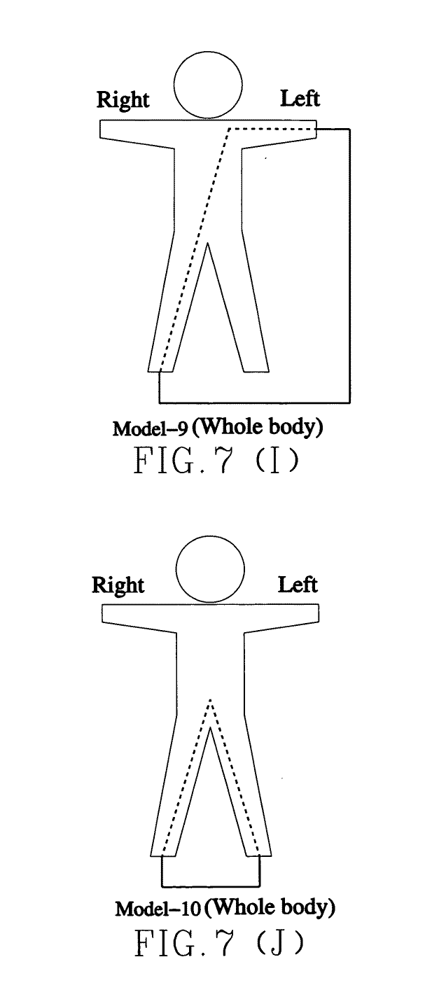

The apparatus body 10 can be any type of body composition measuring device. The apparatus body 10 has at least two anthropometry variables acquiring means for the input of at least, but not limited to, two of the anthropometry variables of the age, body height, body weight and bioelectrical impedance values of the testee. According to the present preferred embodiment, the anthropometry variables acquiring means include a body weight value input unit 11, a number of button input units 12 and a bio-impedance measuring circuit 13. The body weight value input unit 11 is adapted for measuring the body weight of the testee or for allowing the testee to input his (her) body weight. The button input units 12 are adapted for allowing the testee to input his (her) sex, age, body height or other anthropometry variables. The bio-impedance measuring circuit 13 is adapted for measuring the testee's bioelectrical impedance values. According to the present preferred embodiment, as shown in FIGS. 7A-7J, the bio-impedance measuring circuit 13 can measure the bioelectrical impedance values of the testee's whole body, left leg, left arm, right leg and right arm through a current application path (indicated by the imaginary line) and a voltage measuring path (indicated by the real line). The structures and circuits of the aforesaid anthropometry variables acquiring means are of the known art. No further detailed description in this regard is necessary.

The processing unit 20 can be any type of arithmetic logic unit (such as: microprocessor) mounted inside the apparatus body 10. According to the present preferred embodiment, the processing unit 20 has built therein a back propagation-artificial neural network (BP-ANN) for male 30 and a back propagation-artificial neural network (BP-ANN) for female 40. According to the present preferred embodiment, the processing unit 20 selects the corresponding neural network subject to the sex inputted by the testee.

Before description of the detailed architecture of the aforesaid neural networks 30;40, the establishment of these two neural networks 30;40 is explained hereinafter.

At first, the said establishment was to collect and measure the data of the age, body height, body weight, bioelectrical impedance values and fat free mass (FFM) of several healthy males (females), wherein a bioimpedance analyzer, QuandScan 4000 from Bodystat was used to measure the bioelectrical impedance values of the males (females) subject to the method shown in FIG. 7A. Further, a dual energy X-ray absorptiometry (DEXA) from GE was used to scan the whole body of each of males (females), thereby obtaining their body composition data (including fat free mass and fat mass) for reference.

Thereafter, the age, body height, body weight and bioelectrical impedance values of the males (females) were obtained as the network input for the selected artificial neural network, and then the fat free mass of these males (females) were used as the corresponding network output, so that the training of the selected artificial neural network was started. The aforesaid network input were processed through the calculations with initial weight and bias each set to a random value and specific transfer function (Log-Sigmoid or Hyperbolic Tangent Sigmoid) to adjust the weight and bias values till convergence subject to the application of back propagation and Levenberg-Marquardt algorithm, thereby obtaining the optimal weight and bias values.

After the aforesaid training, the back propagation-artificial neural networks (BP-ANN) 30 and 40 were obtained. These two back propagation-artificial neural networks (BP-ANN) 30 and 40 are substantially similar, each comprising an input layer 31 or 41, 1-10 hidden layers 33 or 43, and an output layer 36 or 46.

According to the present preferred embodiment, the input layer 31 or 41 has four input neurons 32 or 42 for receiving the testee's age, body height, body weight and bioelectrical impedance values respectively.

The 1-10 hidden layers 33 or 43 each have 1-15 hidden neurons 34 or 44 and transfer functions 35 or 45 corresponding to the hidden neurons 34 or 44. According to the present preferred embodiment, the transfer functions 35 or 45 can be Log-Sigmoid or Hyperbolic Tangent Sigmoid.

The output layer 36 or 46 comprises an output neuron 37 or 47 and a linear transfer function 38 or 48, and is adapted for outputting the fat free mass (FFM) of the testee. The said fat free mass can be further calculated by the processing unit 20 to obtain the amount and ratio of testee's body fat. The values of the amount and ratio of the testee's body fat can be then displayed on the display unit 50 that is connected to the processing unit 20.

In actual practice, the anthropometric variables inputted by the testee or measured from the testee are transmitted to the back propagation-artificial neural network (BP-ANN) for male 30 or back propagation-artificial neural network (BP-ANN) for female 40 of the processing unit 20 subject to the sex of the testee. For example, if the testee is a male, the anthropometry variables will be calculated by the back propagation-artificial neural network (BP-ANN) for male 30 to obtain the fat free mass of the testee, and thereafter the processing unit 20 can further figure out the testee's body fat.

In order to prove that the invention is more accurate than the conventional linear regression equation, the aforesaid male (female) anthropometry variables were used, with fat free mass determined by DEXA as reference for comparison, to establish a male linear regression equation (1) and a female linear regression equation (2) respectively as follows:

FFM=3.097+7084.41 h²/z+0.150 w+0.00106 gae (1)

FFM=8.674+5846.033 h²/z+0.0762 w+0.0109 age (2)

where:

h: body weight (m)

w: body weight (kg)

age: age (year)

z: bioelectrical impedance value (ohm)

FFM: fat free mass (kg)

The male (female) anthropometry variables were put into equation (1) and equation (2), the correlation coefficient (R) of equation (1) and DEXA on evaluating FFM was R=0.96 and standard deviation (in comparison with the FFM measured by DEXA) SD=2.48 kg; the correlation coefficient (R) of equation (2) and DEXA on evaluating FFM was R=0.90 and standard deviation (in comparison with the FFM measured by DEXA) SD=2.16 kg.

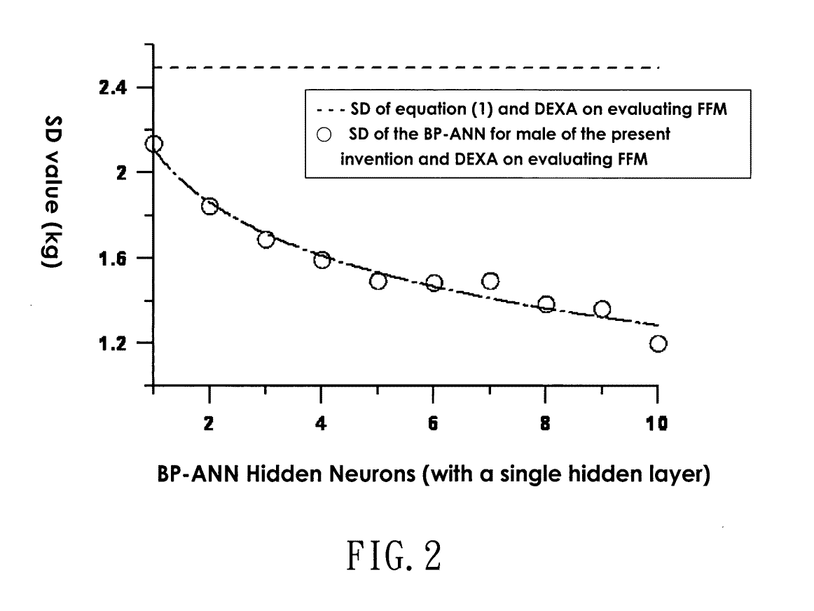

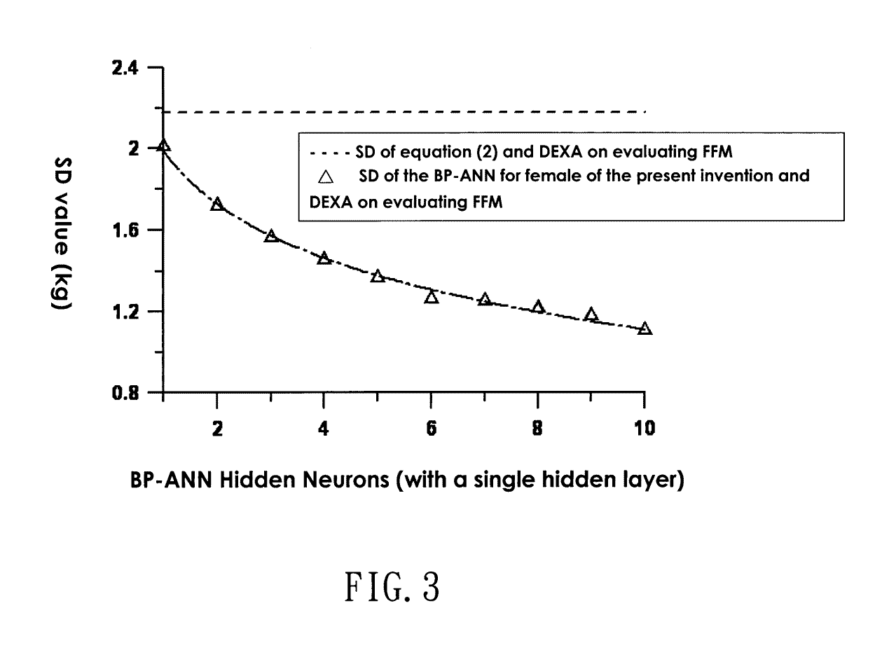

On the contrary, as shown in FIGS. 2 and 3, after comparison between the FFM value measured by the back propagation-artificial neural network (BP-ANN) for male 30 or back propagation-artificial neural network (BP-ANN) for female 40 that has one single hidden layer and the FFM value measured by DEXA, the standard deviation corresponding to 1-10 hidden neurons were smaller than that of the conventional linear regression equation. Further, as shown in the drawings, following increase of the number of hidden neurons (to about 10), the standard deviation (in comparison with the FFM measured by DEXA) approached to a constant. When the number of hidden neurons reached 15, the variation of the standard deviation became insignificant and therefore they were not shown in the drawings.

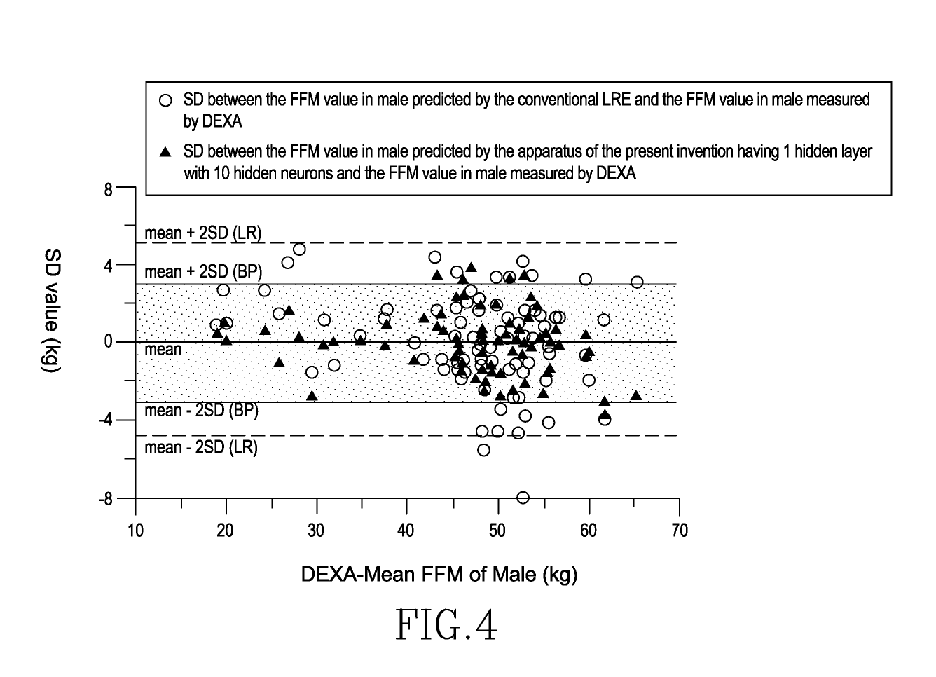

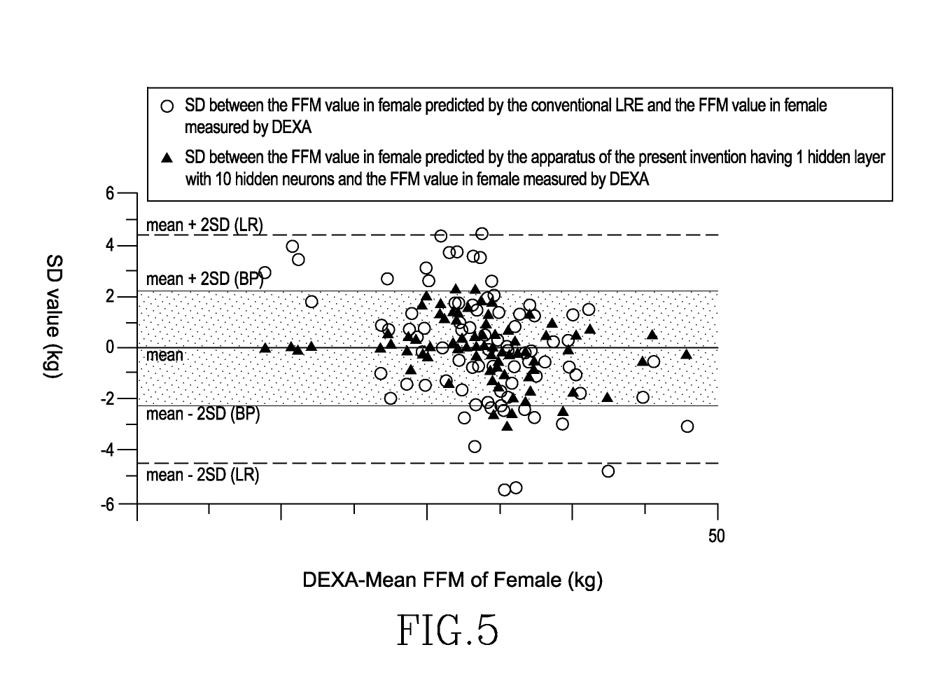

Further, in FIGS. 4 and 5, ▴ represented the standard deviation between the FFM value in male (female) predicted by a body composition measuring apparatus having one single hidden layer with 10 hidden neurons and the FFM value in male (female) measured by DEXA; ◯ represented the standard deviation between the FFM value in male (female) predicted by the conventional linear regression equation and the FFM value in male (female) measured by DEXA. From the distribution of ▴ and ◯ in the drawings, it is apparent that the measured result of the present invention is more accurate than the conventional linear regression equation.

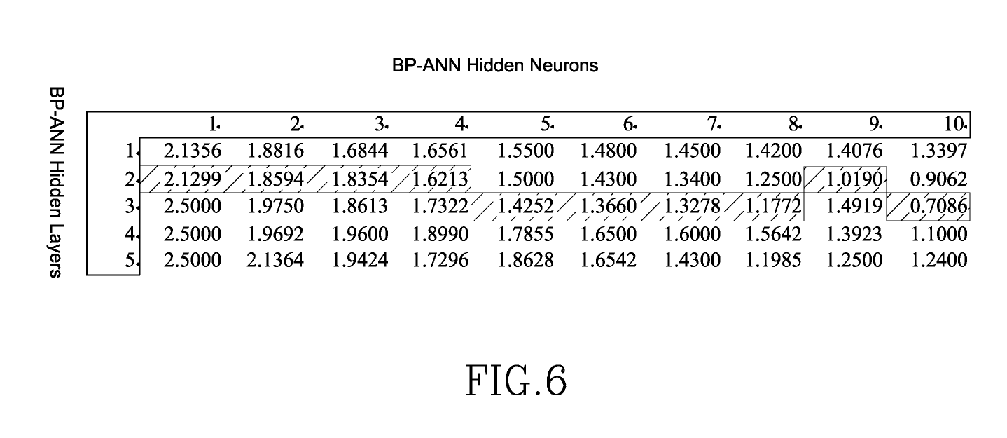

The comparison in FIGS. 2-5 is based on an artificial neural network having one single hidden layer. It is to be understood that, as shown in FIG. 6, the number of the hidden layers in the artificial neural network can be increased to 2-5 layers. If the number of the hidden neuron is 1-10, the corresponding standard deviation (in comparison with the FFM measured by DEXA) are almost all superior to the standard deviation of the conventional linear regression equation.

Generally speaking, as shown in FIG. 6, the optimal number of hidden layers is 2-3 layers (indicated by the marked columns). Further, it is to be understood that following increase of the number of hidden layers (from 5 to 10) in the apparatus of the present invention, the corresponding standard deviation gradually approached a constant. When the number of hidden layers reached 10, the variation of the standard deviation became insignificant, and therefore the values were not indicated in FIG. 6. Further, in view of the fact that the processors of commercial body composition measuring instruments are not high level processors with a limited memory capacity, the number of the hidden layer is preferably within 1-5 layers, or most preferably within 2-3 layers, and the number of hidden neuron is preferably within 1-10.

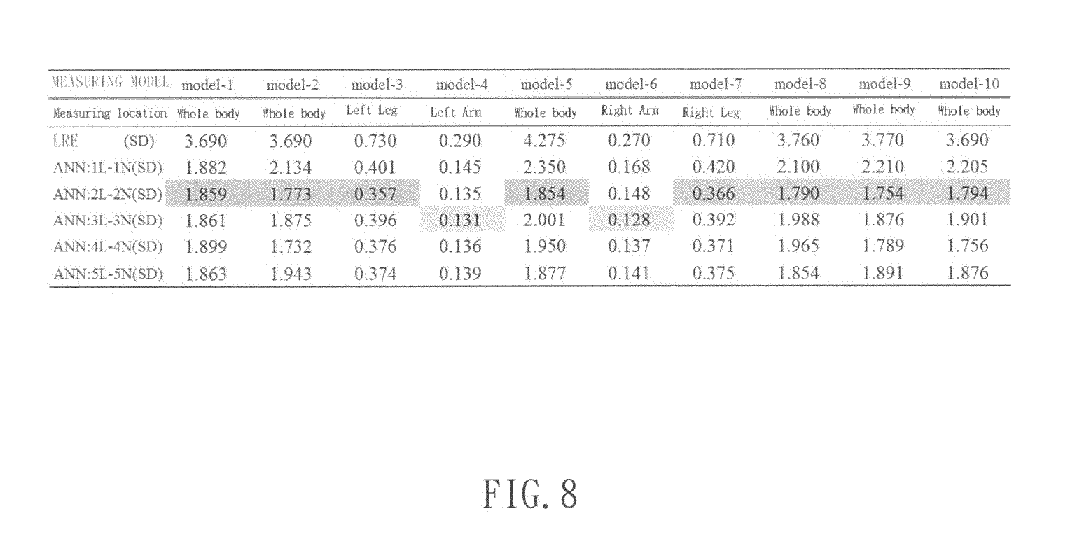

Further, it is to be understood that the experiment data shown in FIGS. 2-6 were based on the bioelectrical impedance values measured by the measurement model shown in FIG. 7(A). However, this measurement model is not a limitation. Referring to FIGS. 7(A)-7(J) and FIG. 8, the bioelectrical impedance values measurement models shown in FIGS. 7(B)-7(J) are applicable to the establishment of the artificial neural network of the present invention. FIG. 8 shows an artificial neural network established subject to bioelectrical impedance values on each limb for predicting the FFM of each limb. As indicated by the data shown in FIG. 8, the standard deviation (SD) obtained from different artificial neural network models of the present invention were all smaller than the standard deviation (SD) obtained from the conventional linear regression equation, wherein the number of hidden layer 1-5 layers showed better and 2-3 layers showed the best as indicated by the marked columns. Therefore, the invention is applicable for the measurement of every limb, showing high accuracy.

Further, during establishment of the artificial neural network of the present invention, except the anthropometry variables of age, body height, body weight and bioelectrical impedance values, other anthropometry variables (such as waist, hip, menstrual cycle, etc.) may be added to increase the measuring accuracy. Further, it is not imperative to classify the built-in artificial neural network for male or female use, in another word, one single artificial neural network can be established for both the males and the females that can show better accuracy than the linear regression model.

In conclusion, the invention utilizes the easily obtained anthropometry variables to establish a specific artificial neural network. When compared to the conventional linear regression equation, the invention shows high accuracy, and may not need to measure so much anthropometry variables. Further, the measuring speed and the operating speed of the invention reach a certain level, practical for use in home use and medical grade human body composition measuring equipments.

Although a particular embodiment of the invention has been described in detail for purposes of illustration, various modifications and enhancements may be made without departing from the spirit and scope of the invention. Accordingly, the invention is not to be limited except as by the appended claims.

What is claimed is:

1. A body composition measuring apparatus using a bioelectric impedance analysis associated with a neural network algorithm, comprising:
an apparatus body, said apparatus body comprising at least one anthropometry variables acquiring means for obtaining anthropometry variables of a testee, including at least two of the age, body height, body weight and bioelectrical impedance values of the testee; and a processing unit mounted inside said apparatus body and connected to said at least one anthropometry variables acquiring means, said processing unit having built therein at least one back propagation-artificial neural network (BP-ANN), each said back propagation-artificial neural network comprising: an input layer, said input layer comprising a plurality of input neurons adapted for receiving said anthropometry variables from said at least one anthropometry variables acquiring means; 1-10 hidden layers, each said hidden layer comprising 1-15 hidden neurons and a transfer function corresponding to each said hidden neuron, each said transfer function being a Log-Sigmoid or Hyperbolic Tangent Sigmoid; and an output layer, said output layer comprising an output neuron and a linear transfer function for outputting fat free mass (FFM), said fat free mass (FFM) being processable by said processing unit to obtain the body fat of the testee.

2. The body composition measuring apparatus using a bioelectric impedance analysis associated with a neural network algorithm as claimed in claim 1, wherein the number of said hidden layers is 1-5 layers.

3. The body composition measuring apparatus using a bioelectric impedance analysis associated with a neural network algorithm as claimed in claim 2, wherein the number of the hidden neuron of each said hidden layer is 1-10.

4. The body composition measuring apparatus using a bioelectric impedance analysis associated with a neural network algorithm as claimed in claim 1, wherein the number of the hidden layers is 2-3 layers.

5. The body composition measuring apparatus using a bioelectric impedance analysis associated with a neural network algorithm as claimed in claim 4, wherein the number of the hidden neuron of each said hidden layer is 1-10.

6. The body composition measuring apparatus using a bioelectric impedance analysis associated with a neural network algorithm as claimed in claim 1, wherein said at least one back propagation-artificial neural network (BP-ANN) uses the age, body height, body weight and bioelectrical impedance values of a number of persons as the network input for training, and the fat free mass of said persons as a network output for training.

7. The body composition measuring apparatus using a bioelectric impedance analysis associated with a neural network algorithm as claimed in claim 1, wherein said at least one back propagation-artificial neural network (BP-ANN) uses Levenberg-Marquardt algorithm to run training.

8. The body composition measuring apparatus using a bioelectric impedance analysis associated with a neural network algorithm as claimed in claim 1, wherein the number of said at least one back propagation-artificial neural network (BP-ANN) is 2, including one back propagation-artificial neural network for measuring male testees and the other back propagation-artificial neural network for measuring female testees.

9. The body composition measuring apparatus using a bioelectric impedance analysis associated with a neural network algorithm as claimed in claim 1, wherein said at least one anthropometry variables acquiring means comprises a bio-impedance measuring circuit adapted for measuring said bioelectrical impedance values, said bioelectrical impedance values being selected from the group consisting of the impedance of the whole body, the impedance of the left leg, the impedance of the left arm, the impedance of the right leg and the impedance of the right arm.

10. The body composition measuring apparatus using a bioelectric impedance analysis associated with a neural network algorithm as claimed in claim 9, wherein the number of the hidden layer is 1-5 layers.

11. The body composition measuring apparatus using a bioelectric impedance analysis associated with a neural network algorithm as claimed in claim 10, wherein the number of the hidden layer is 2-3 layers.
